(19) United States
(12) Patent Application Publication
(10) Pub. No.: US 2026/0325740 A1
(43) Pub. Date: Oct. 22, 2026

(54) MEDICAL DEVICE PACKAGING AND RELATED METHODS

(71) Applicant: Regeneron Pharmaceuticals, Inc., Tarrytown, NY (US)

(72) Inventors: Bryan C. Grygus, Clifton Park, NY (US); Jim Fedor, Bayport, MN (US); Wayne Phillips, Hudson, WI (US); Anne Wespetal, Menomonie, WI (US); Ashley Johannes, Halfmoon, NY (US); Tanya Magana, New Richmond, WI (US)

(73) Assignee: Regeneron Pharmaceuticals, Inc., Tarrytown, NY (US)

(21) Appl. No.: 19/728,421

(22) Filed: Jul. 2, 2026

Related U.S. Application Data
(63) Continuation of application No. 18/744,094, filed on Jun. 14, 2024, now Pat. No. 12,691,213.
(60) Provisional application No. 63/508,812, filed on Jun. 16, 2023.

(51) Int. Cl. A61M 5/00

(52) U.S. Cl. CPC A61M 5/002

(57) ABSTRACT

A packaging comprising a tray having a body surrounding an exposed cavity and a groove; a lip surrounding the opening, wherein the lip extends radially outward from the cavity and defines a periphery of the tray; a retainer for covering a portion of the opening of the tray, the retainer having an aperture, wherein the aperture corresponds to the groove of the tray; a removable cover; and an auto-injector contained within the cavity, wherein the auto-injector is pre-filed with a medicament or other fluid.

### CROSS-REFERENCE TO RELATED APPLICATIONS

This application is a continuation of U.S. application Ser. No. 18/744,094, filed Jun. 14, 2024, which claims priority to U.S. Provisional Patent Application No. 63/508,812, filed Jun. 16, 2023, which is incorporated by reference herein.

### FIELD OF THE DISCLOSURE

Various embodiments of the present disclosure relate to packaging for a medical device. Embodiments of the disclosure may include a packaging for housing a drug delivery device, e.g., an auto-injector.

### INTRODUCTION

Many medical devices are packaged for distribution to healthcare facilities and/or for storage, for example. Once a user, e.g., a healthcare provider, is ready to use the medical device, the medical device is removed from the packaging and prepped, if necessary, for use on a patient. In order to remove medical devices from packaging, users often need to directly handle the devices and to push, pull, lift, slide, or otherwise directly touch the medical device with their hands. In some instances, it may not be desirable to handle the medical devices too much, e.g., for reasons of sterility or to inhibit damage to the medical devices, when removing the medical devices from their packaging. For example, auto-injectors may be contaminated when they are handled during removal of packaging, which may jeopardize the sterility of the components of the auto-injector, e.g., a chamber, syringe, needle, needle attachment, and/or any medicament or other fluid contained within.

### SUMMARY

In one aspect, the present disclosure describes a packaging comprising a tray having a body surrounding an exposed cavity and a groove; a lip surrounding the opening, wherein the lip extends radially outward from the cavity and defines a periphery of the tray; a retainer for covering a portion of the opening of the tray, the retainer having an aperture, wherein the aperture corresponds to the groove of the tray; a removable cover; and an auto-injector contained within the cavity, wherein the auto-injector is pre-filed with a medicament or other fluid.

Various embodiments of the packaging may include one or more of the following aspects. The packaging may include a gaseous sterilant. The packaging may include a gaseous sterilant including one or more of ethylene oxide, vaporized hydrogen peroxide, ethyl alcohol, nitrous oxide, or nitrogen dioxide. The tray may include a first plurality of sidewalls and a second plurality of sidewalls. The packaging may include a lug. The lip may include an adhesive and may form a seal with the retainer to enclose the auto-injector within the cavity. The tray and/or the retainer may include one or more of polypropylene, polystyrene, polyethylene, polycarbonate, polyvinyl chloride, or polyethylene terephthalate glycol. The removable cover may include high-density polyethylene fibers, thermoplastic materials, or a combination thereof. The medicament or fluid may comprise one or more of dupilumab and cemiplimab.

In another aspect, the present disclosure describes a packaging for an auto-injector, the packaging comprising a tray having an opening and a cavity; a lip surrounding the opening, wherein the lip extends radially outward from the cavity and defines a periphery of the tray; a removable cover having a periphery that is adhered to the lip; and a retainer for covering a portion of the opening of the tray.

Various embodiments of the packaging may include one or more of the following aspects. The removable cover may be permeable to a gaseous sterilant. The gaseous sterilant may include one or more of ethylene oxide, vaporized hydrogen peroxide, ethyl alcohol, nitrous oxide, or nitrogen dioxide. The tray and/or of the retainer may include one or more of polypropylene, polystyrene, polyethylene, polycarbonate, polyvinyl chloride, or polyethylene terephthalate glycol. The removable cover may include high-density polyethylene fibers, thermoplastic materials, or a combination thereof. The auto-injector may contain a medicament comprising one or more of dupilumab and cemiplimab.

In another aspect, the present disclosure describes a method of removing an externally-sterilized auto-injector from a packaging, the packaging including a tray having an opening and a cavity, a retainer, a removable cover, and a gaseous sterilant, the method comprising: removing the removable cover to expose the retainer; removing the retainer to expose the cavity of the tray; grasping a portion of the externally-sterilized auto-injector held within the cavity of the tray; and ejecting the externally-sterilized auto-injector from the tray.

Various embodiments of the method may include one or more of the following aspects. The method may include pushing a portion of the tray towards the cavity of the tray, wherein pushing the portion of the tray causes a portion of the tray to flex. Pushing the portion of the tray may cause the externally-sterilized auto-injector to release from the cavity of the tray. The gaseous sterilant may include one or more of ethylene oxide, vaporized hydrogen peroxide, ethyl alcohol, nitrous oxide, or nitrogen dioxide. The externally-sterilized auto-injector may contain a medicament comprising one or more of dupilumab and cemiplimab

### BRIEF DESCRIPTION OF THE DRAWINGS

The accompanying drawings, which are incorporated into and constitute a part of this specification, illustrate various exemplary embodiments and, together with the description, serve to explain the principles of the disclosed embodiments. The drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

There are many inventions described and illustrated herein. The described inventions are neither limited to any single aspect nor embodiment thereof, nor to any combinations and/or permutations of such aspects and/or embodiments. Moreover, each of the aspects of the described inventions, and/or embodiments thereof, may be employed alone or in combination with one or more of the other aspects of the described inventions and/or embodiments thereof. For the sake of brevity, certain permutations and combinations are not discussed and/or illustrated separately herein. Notably, an embodiment or implementation described herein as “exemplary” is not to be construed as preferred or advantageous, for example, over other embodiments or implementations; rather, it is intended reflect or indicate the embodiment(s) is/are “example” embodiment(s).

FIG. 1 provides a perspective view of an exemplary packaging, according to an embodiment of the present disclosure.

FIG. 2 provides a perspective of an exemplary tray, according to an embodiment of the present disclosure.

FIGS. 3A-3H provide detailed views of an exemplary tray, according to embodiment of the present disclosure.

FIG. 3I provides a top view of an exemplary tray, according to an embodiment of the present disclosure.

FIG. 4 provides a perspective view of an exemplary retainer, according to an embodiment of the present disclosure.

FIGS. 5A-5E provide detailed views of an exemplary retainer, according to an embodiment of the present disclosure.

FIG. 6 provides a detailed view of an exemplary removable cover, according to an embodiment of the present disclosure.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items. Further, as used herein, the terms “about,” “substantially,” and “approximately” generally mean +/−10% of the indicated value.

As used in the present disclosure, the term “sterilization” refers to achieving a level of sterility appropriate for a formulated drug substance or drug product for commercial distribution and use. More particularly, “sterilization” as used herein includes, but is not limited to, achieving a level of sterility that sterilizes a drug delivery device while packaged within, e.g., a packaging according to aspects of the present disclosure, for commercial distribution and use. Such a level of sterility may be defined in, for example, regulatory guidelines or regulations, such as guidelines released by the U.S. Food and Drug Administration. In some embodiments, such a level of sterility may include, for example, a 6-log reduction in microbial populations of biological indicators placed on an outside or inside surface of a drug product (e.g., an outside surface of an auto-injector or an inside surface of a blister pack). In other embodiments, such a level of sterility may include, for example, a 9-log or 12-log reduction in microbial populations of biological indicators. Sterilization refers to achieving such an appropriate level of sterility while also achieving a sufficiently low level of residual sterilizing chemicals (e.g., vaporized hydrogen peroxide, ethylene oxide, nitrogen dioxide (NO₂), nitric oxide (NO), etc.) for commercial distribution and use. Such a low level of residual sterilizing chemical may also be defined in regulatory guidelines or regulations.

As used in the present disclosure, the term “external sterilization” refers to the sterilization of a drug delivery device in a container or packaging, such as in a primary packaging component, or in both primary and secondary packaging components, suitable for commercial distribution and use. External sterilization may include, but is not limited, to terminal sterilization, as readily understood by those of ordinary skill in the art.

### DETAILED DESCRIPTION

Embodiments of the present disclosure relate to medical device packaging, and, in particular, to packaging for auto-injectors. In some embodiments, instead of a functional auto-injector, it could be a training device that is identical in outer dimensions but is otherwise not able to deliver a medicament. In some embodiments, the packaging may be configured to contain externally-sterilized (or terminally-sterilized) auto-injectors. In particular, embodiments of the disclosure may be directed to packaging for externally-sterilized (or terminally-sterilized) auto-injectors containing medicaments or other fluids (e.g., placebo). Exemplary auto-injectors may be pre-filled with, e.g., 50 mL or less, 20 mL or less, 15 mL or less, 10 mL or less, 5 mL or less, or 1 ml or less of an medicament or other fluids e.g., (placebo). Exemplary auto-injectors are described in U.S. Pat. Nos. 10,182,969 B2, 11,406,565 B2, US 2022/0401302A1, WO 2018/204779 A1, and WO 2023/091922 A2, which are incorporated by reference herein in their entireties. The packaging may include one or more features to facilitate removal of a medical device from the packaging. For example, the packaging may include features that allow a user to pull the auto-injector up and out of the packaging with minimal disturbance to the auto-injector or its plunger/needle, and/or impact on the sterility of the auto-injector. The packaging may include one or more deformable projections or portions that may be inverted, pushed, and/or otherwise deformed in order to dislodge, eject, push, or otherwise move the medical device to release it from the packaging. The packaging may also be flexible to allow a user to push on the projection to bend or flex the packaging to promote release of the medical device contained within.

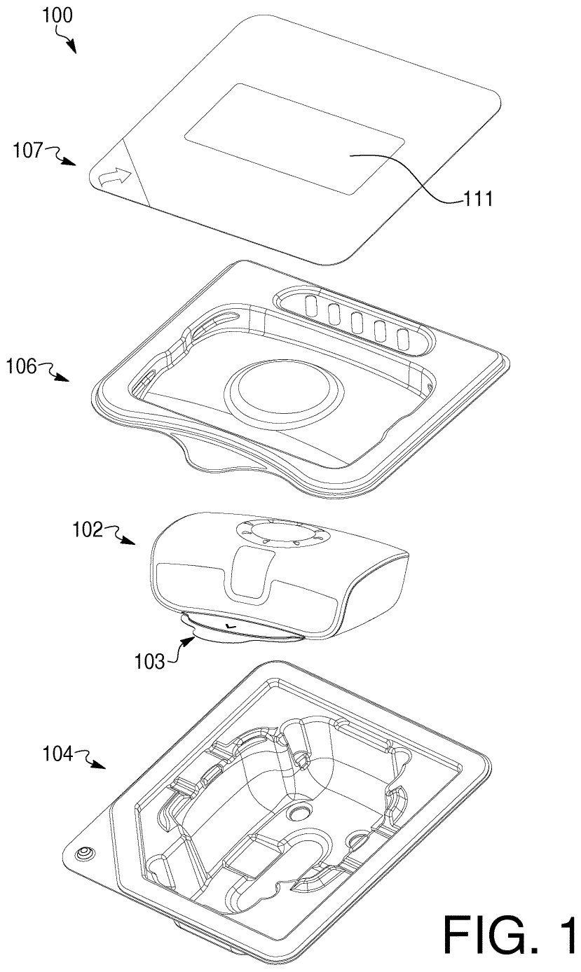

FIG. 1 depicts components of an exemplary packaging 100 for a medical device 102. Specifically, FIG. 1 depicts a tray 104, a retainer 106, and a removable cover 107. Each component of packaging 100 will be discussed in further detail. As shown in FIG. 1, medical device 102 may fit into an opening of tray 104 and retainer 106 may be configured to be secured to a portion of tray 104 via a friction fit, such that tray 104 and retainer 106 form an enclosure around medical device 102 and close an opening of tray 104. Removable cover 107 may be configured to attach to a portion of tray 104, e.g., via an adhesive. While the figures depict aspects where tray 104 and retainer 106 are formed as discreet components configured to be complementary to one another, those of ordinary skill in the art will readily recognize that the tray and retainer may be made of a suitable unitary or one-piece construction. In such embodiments, packaging 100 may include a hinge, e.g., a joint, fulcrum, or any appropriate connector. In such embodiments, retainer 106 may be connected to tray 104 via a hinge allowing retainer 106 to rotate about the hinge connection to open or close the opening of tray 104, e.g., in a clamshell configuration. In further aspects, the hinge connection feature may be selectively frangible.

To facilitate removal of medical device 102 from packaging 100, a user may remove removable cover 107 from a portion of tray 104, and then remove retainer 106 from a portion of tray 104, exposing medical device 102. A user may then orient tray 104 such that an opening of tray 104 is angled toward a surface onto which medical device 102 is to be ejected. Medical device 102 may then be ejected from tray 104. A user may also push on and/or flex a portion of tray 104 to eject medical device 102 from tray 104. For example, the user may push on and/or flex a portion of tray 104 and turn tray 104 sideways or up-side-down in order to discharge medical device 102 from tray 104 and onto a sterile surface, e.g., a sterile table, tray, cloth, liner, etc. Flexure of tray 104 while an opening of tray 104 is angled gravitationally downward may cause medical device 102 to be released, e.g., ejected, out of the packaging onto a surface with the user contacting only the packaging and not directly contacting medical device 102.

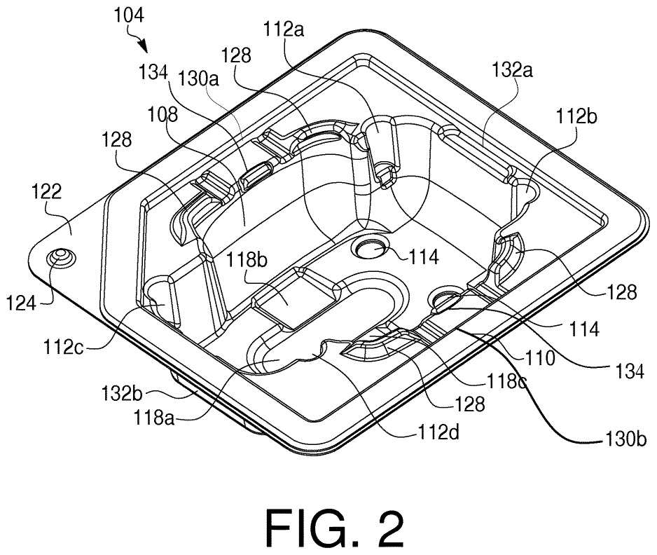

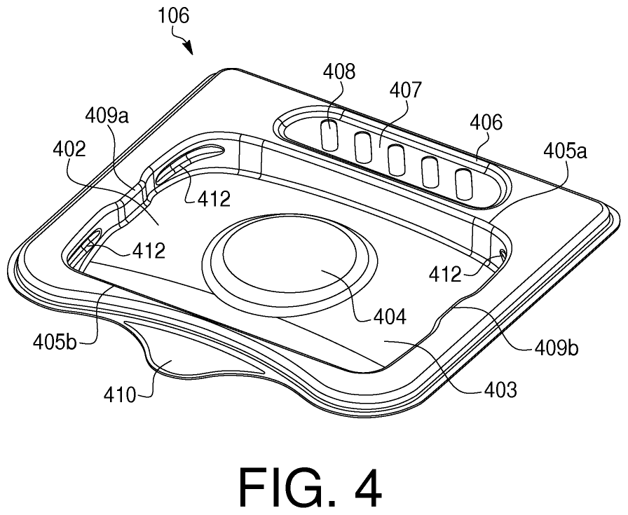

Packaging 100 may include tray 104, in which the auto-injector is contained during storage, and a lip 110 extending around a perimeter of tray 104 and defining an opening in tray 104. The top of tray 104 (shown, e.g., in FIGS. 1 and 2) may be covered with retainer 106 (shown, e.g., in FIGS. 1 and 4), which may enclose the auto-injector within tray 104 during sterilization and/or storage. In some embodiments, tray 104 and/or retainer 106 may be sealed with a removable cover 107, which may enclose the medical device, e.g., auto-injector, in tray 104 and/or retainer 106 during sterilization and/or storage. In some embodiments, packaging 100 may be blister packaging, i.e., packaging 100 may be a pre-formed plastic packaging. In some examples, packaging 100 and/or components of packaging 100 may be made from a formable web, such as a thermoformed plastic.

Packaging 100 may be suitable for use with an external (or terminal) sterilization process, e.g., an ethylene oxide (EtO), a vapor hydrogen peroxide (VHP) sterilization process and/or an ethyl alcohol sterilization process. Accordingly, a medical device (e.g., an auto-injector) may be packaged within packaging 100 and then subjected to external sterilization or terminal sterilization. Sterilants, such as VHP, may not affect the medication contained within the auto-injector but may sterilize the exterior surface of the auto-injector. Exemplary sterilization processes are described in WO 2018/182929 A1, which is incorporated by reference herein in its entirety. Materials used in packaging 100 may be semi-permeable to sterilizing agents to allow the sterilizing agents to traverse portions of or all of the packaging to sterilize the exterior of the medical device contained within the packaging, as well as the interior of the packaging, once the medical device is sealed within the packaging. In some embodiments, at least 75%, at least 80%, at least 85%, at least 90%, or at least 95%, of packaging 100 is sterilized. For example, removable cover 107 may be permeable to vapor hydrogen peroxide and/or an ethyl alcohol. Removable cover 107 may be formed of, e.g., Tyvek or other suitable high-density polyethylene fibers, ethylene-vinyl acetate, and/or other thermoplastic materials. Removable cover 107 may also be permeable to a suitable gaseous sterilant, such as ethylene oxide, vaporized hydrogen peroxide, ethyl alcohol, nitrous oxide, nitrogen dioxide, or any appropriate sterilant in the art. Components of packaging 100, e.g., tray 104 and/or retainer 106, may be thermoformed plastic. For example, tray 104 and/or retainer 106 may be formed of polypropylene, polystyrene, polyethylene, polycarbonate, polyvinyl chloride, or polyethylene terephthalate glycol (PETG). Components of packaging 100, e.g., tray 104, retainer 106, and/or removable cover 107, and the size and/or shape of such components may be configured to facilitate high speed manufacturing of packaging 100 and/or improve material distribution of packaging 100.

In some aspects of the present disclosure, packaging 100, e.g., one or more of tray 104, retainer 106, and removable cover 107, discussed herein may be impermeable (or partially permeable or partially impermeable) to one or more suitable gaseous sterilants. In such aspects, it is contemplated that a drug delivery device (e.g., the auto-injector of the present disclosure) may be appropriately packaged within the packaging (e.g., the tray and retainer) of the present disclosure along with a sterilant or source of sterilant (as disclosed in greater detail below), and thereby sealed for delivery by an exemplary removable cover having limited or no permeability to the sterilant. In some embodiments, the sterilant sealed within packaging 100 may be in a gaseous, solid, liquid form, or a combination thereof. Still further, it is contemplated that a source of sterilant may be sealed within packaging 100, wherein the source of sterilant may be a solid apparatus configured to release gaseous sterilant upon a predetermined triggering event or upon the passing of a predetermined time delay. In some aspects, the solid apparatus may include multiple layers or chambers storing ingredients configured to combine and produce a gaseous sterilant, e.g., ethylene oxide, vaporized hydrogen peroxide, ethyl alcohol, nitrous oxide, nitrogen dioxide, or any appropriate sterilant in the art. Still further, the solid apparatus may include a sterilant wafer containing a polymer configured to generate and/or release a suitable gaseous sterilant after packaging 100 has been sealed. Those of ordinary skill in the art will readily recognize that the suitable gaseous sterilant may include any of the gaseous sterilants described herein. In some embodiments, packaging 100, e.g., one or more of tray 104, retainer 106, and removable cover 107, may include an indicator to allow a user to determine whether a component of packaging 100, e.g., one or more of tray 104, retainer 106, and removable cover 107, and its contents have been sterilized. For example, tray 104, retainer 106, and/or removable cover 107 may include an indicator (e.g., a tape or a label) that changes appearance in color or pattern to visually show whether a sterilization process has been performed.

Designing packaging 100 to be used with external sterilization processes and to allow a user to remove the contents of the packaging without directly handling the contents, as described herein, reduces the likelihood that sterility of the contained medical device will be compromised. As a result, packaging 100, as described herein, may also reduce the chance that a person on whom the medical device is used will be infected by the medical device.

An externally-sterilized auto-injector may be one in which any viable organisms on the surface of the auto-injector have been terminated or killed, making them non-viable even though they may remain. Any components of the auto-injector, e.g., a needle mechanism, cartridge, body, etc., may be externally-sterilized. In some aspects, depyrogenation and/or other removal processes may be undertaken prior to external sterilization.

As shown in FIG. 1, device 102 may include an adhesive patch 103. Adhesive patch 103 may be configured to couple to a tissue-engaging surface to help secure device 102 to a user's body (e.g., skin). Adhesive patch 103 may be formed from fabric or any other suitable material, and may include an adhesive. The adhesive may be an aqueous or solvent-based adhesive, or may be a hot melt adhesive, for example. Suitable adhesives also include acrylic based, dextrin based, and urethane based adhesives as well as natural and synthetic elastomers. In some examples, the adhesive provided on patch 103 may be activated upon contact with a user's skin. In yet another example, patch 103 may include a non-woven polyester substrate and an acrylic or silicone adhesive. Patch 103 may be joined to device 102 by, e.g., a double-sided adhesive, or by other mechanisms like ultrasonic welding. Adhesive patch 103 may be externally-sterilized, such that packaging 100 protects the sterility of adhesive patch 103 prior to use of device 102.

FIG. 2 depicts a perspective view of tray 104. Tray 104 may include a cavity 108 configured to contain a portion of an auto-injector. Portions of tray 104 may be shaped to correspond to the shape of device 102, as shown in FIG. 1. For example, portions of tray 104 may be configured such that portions of tray 104 form a friction fit with at least some portions of device 102. In other examples, a portion of tray 104 may be configured to form a friction fit with adhesive patch 103 of device 102 (FIG. 1). Tray 104 and/or components of tray 104 may be made from a formable web, such as a thermoformed plastic. In some embodiments, tray 104 and/or components of tray 104 may be formed of a flexible material. For example, tray 104 and/or components of tray 104 may be formed of one or more of polypropylene, polystyrene, polyethylene, polycarbonate, polyvinyl chloride, or polyethylene terephthalate glycol (PETG).

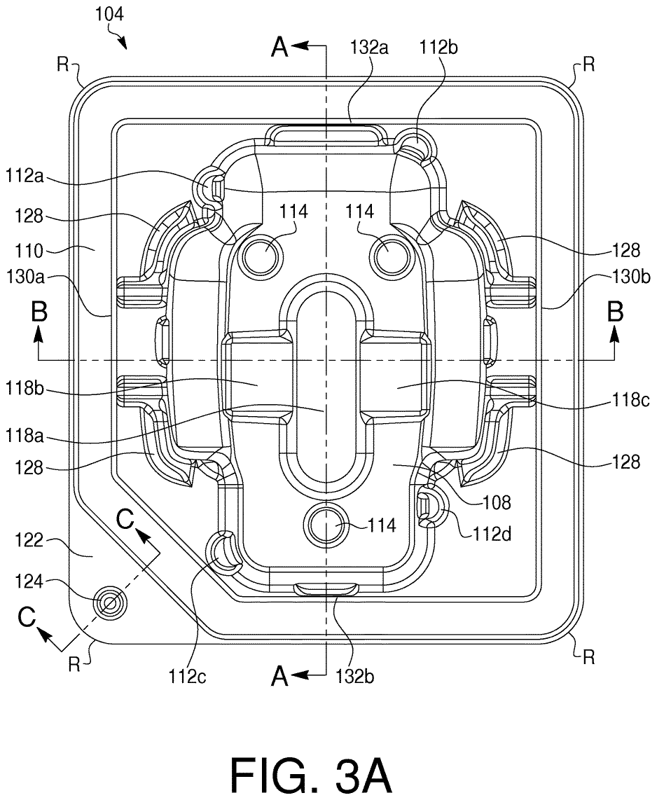

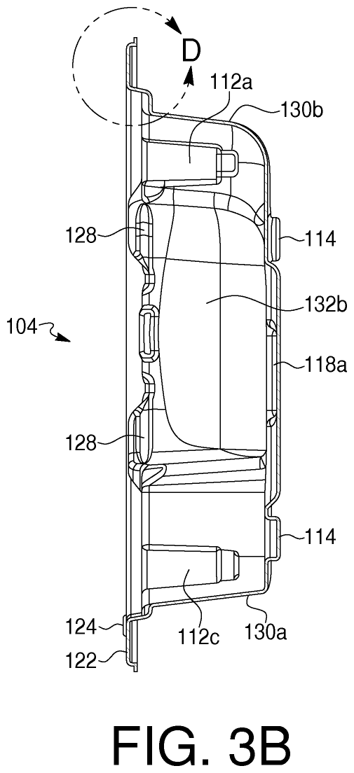

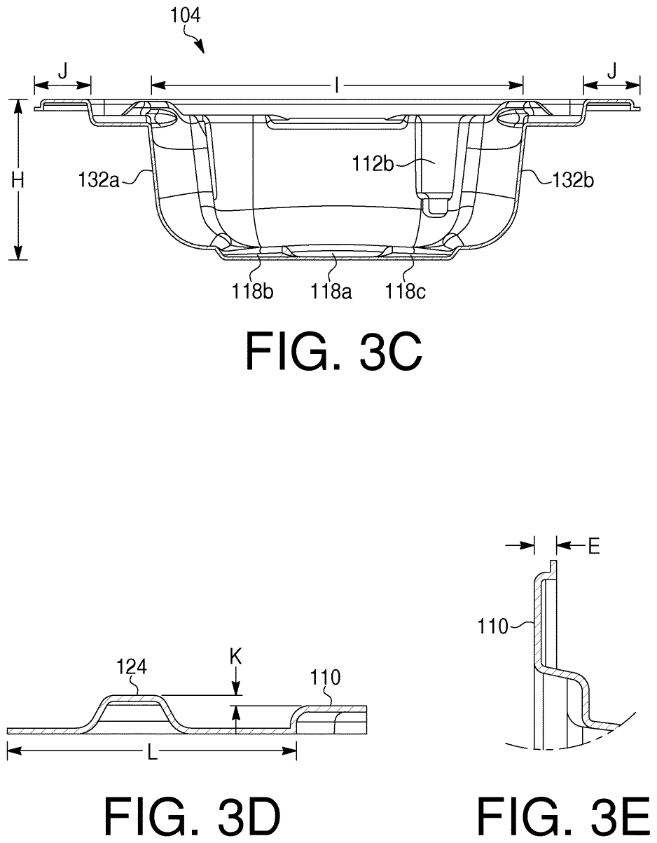

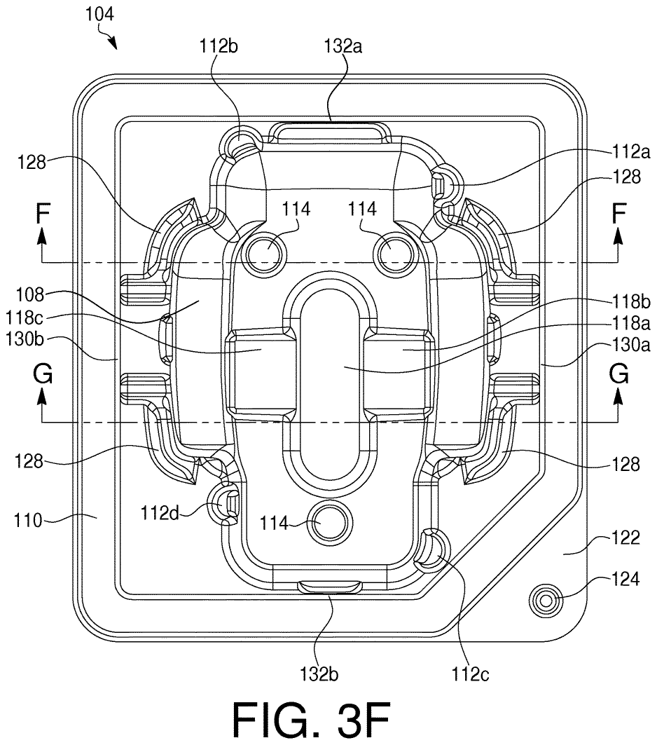

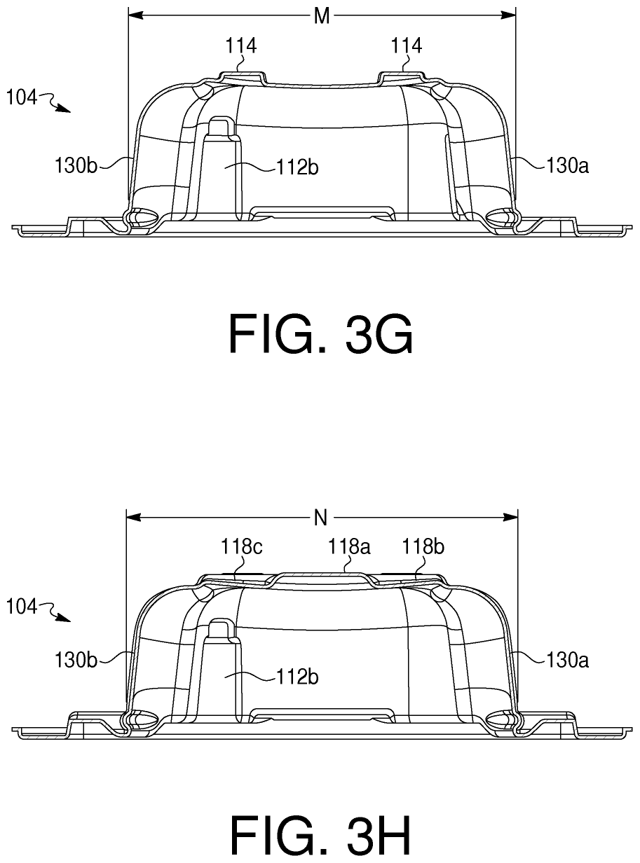

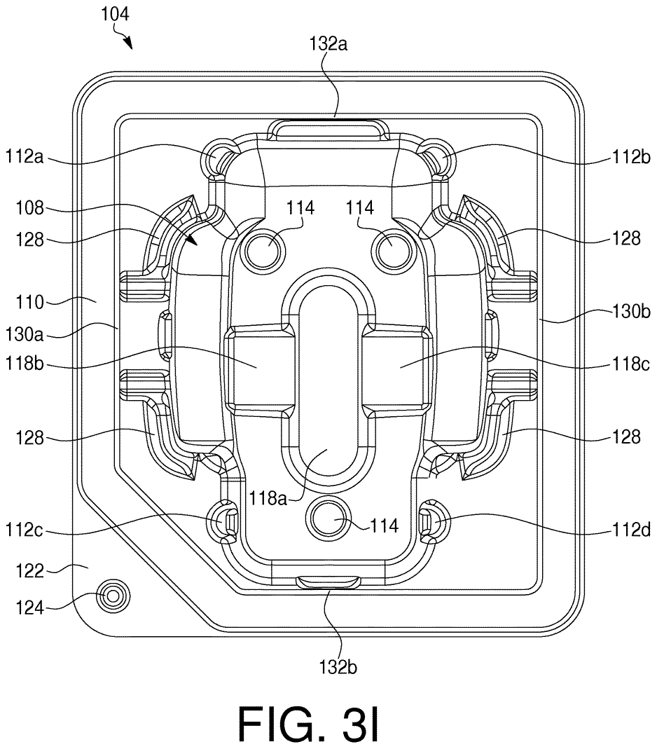

Referring to FIGS. 2, 3A-3C, and 3F-3H, tray 104 may include a plurality of sidewalls. For example, tray 104 may include a first plurality of sidewalls 130a, 130b and a second plurality of sidewalls 132a, 132b. In some embodiments, a length of first plurality of sidewalls 130a, 130b may be larger than a length of second plurality of sidewalls 132a, 132b. In some embodiments, a length of first plurality of sidewalls 130a, 130b may be less than a length of second plurality of sidewalls 132a, 132b. In some embodiments, a length of first plurality of sidewalls 130a, 130b may be equal to a length of second plurality of sidewalls 132a, 132b.

Tray 104 may include a plurality of lugs 112a, 112b, 112c, 112d. As shown in FIGS. 2 and 3A, lugs 112a, 112b, 112c, 112d may be shaped to correspond to the shape of device 102, as shown in FIG. 1. In some examples, lugs 112a, 112b, 112c, 112d may be shaped to form a space or gap between a portion of device 102 and tray 104. For example, once a user opens packaging 100 to expose device 102 within tray 104, a user may insert one or more fingers into the space and/or spaces formed by lugs 112a, 112b, 112c, 112d in order to grasp and/or lift device 102 from tray 104. Tray 104 may include at least two lugs, at least three lugs, or at least four lugs. FIGS. 3A and 3I depict lugs 112a, 112b, 112c, 112d in various positions. For example, FIG. 3A depicts a plurality of lugs 112a, 112b, 112c, 112d in offset positions where lug 112a is offset, i.e., not directly across from, lug 112b, and lug 112c is offset from lug 112d. In FIG. 3I, lug 112a is located across from lug 112b and lug 112c is located across from lug 112d. Referring to FIGS. 2 and 3A, first sidewall 130a of first plurality of sidewalls 130a, 130b may include lug 112a and 112c, second sidewall 130a of first plurality of sidewalls 130a, 130b may include lug 112d, and first sidewall 132a of second plurality of sidewalls 132a, 132b may include lug 112b. Referring to FIG. 3I, lug 112a and lug 112c may be located in a first sidewall 130a of first plurality of sidewalls 130a, 130b may include lug 112a and lug 112c and second sidewall 130b of first plurality of sidewalls 130a, 130b may include lug 112b and 112d. Lugs 112a, 112b, 112c, 112d may be positioned in any appropriate location to allow for a user to comfortably and/or easily grasp a portion of device 102 and/or lift device 102 from tray 104. Lugs 112a, 112b, 112c, 112d may also provide structural stability to tray 104 and/or additional space for the sterilant to circulate within tray 104 and/or around medical device 102. In other embodiments, lug 112a, 112b, 112c, 112d may aid in the manufacturing process of tray 104.

In some embodiments, tray 104 may include at least one indent 114. Indent 114 may be shaped to correspond to a portion of device 102. Indent 114 may have a generally circular or round shape. As shown in FIG. 3A, tray 104 may include a plurality of indents 114, for example, at least one indent 114, at least two indents 114, or at least three indents 114. Indent 114 may be placed at any appropriate location of tray 104 such that indent 114 properly fits against and/or corresponds to a portion of device 102. FIG. 3G depicts an exterior side view of tray 104 wherein each indent 114 projects outwards from the base of tray 104 away from cavity 108 and device 102 when device 102 is received in tray 104. In some embodiments, indent 114 may aid in the manufacturing process of tray 104, e.g., indent 114 may serve as an ejection surface from a mold.

In some embodiments, tray 104 may include a depression 118a, 118b, 118c on a bottom portion of tray 104. Depression 118a, 118b, 118c may be shaped to correspond to a portion of device 102, e.g., a drop pin of an auto-injector, which may also be referred to as an anti-firing mechanism. As shown in FIG. 3A, one of the depressions, e.g., depression 118a, may have a generally oval shape. In some embodiments, as shown in FIG. 3A, depression 118b may be located on one side of depression 118a and depression 118c may be located on an opposite side of depression 118a. In some embodiments, depression 118a, 118b, 118c may be flexible and may be configured to facilitate removal of device 102 from tray 104. Depression 118a, 118b, 118c may be associated with the base of tray 104 so that projection 116 is oriented beneath a device when device 102 is housed within tray 104 and packaging 100. Depression 118a, 118b, 118c may be made of a compressible material that deforms under pressure, e.g., pressure applied by a user. Depression 118a, 118b, 118c may be made of the same material as the rest of tray 104 or may be formed of a different material, e.g., a relatively more flexible material. In some embodiments, as discussed above, depression 118a, 118b, 118c and tray 104 may be thermoformed plastic. For example, depression 118a, 118b, 118c and/or tray 104 may be formed of one or more of polypropylene, polystyrene, polyethylene, polycarbonate, polyvinyl chloride, or polyethylene terephthalate glycol (PETG).

In some embodiments, packaging 100 may be configured for an auto-injector including a button or switch on a bottom surface of the auto-injector. In such embodiments, tray 104 may be configured such that the auto-injector securely fits into a portion of tray 104, e.g., at least one depression 118a, 118b, 118c, in order to prevent the button or switch from turning on, e.g., due to movement of the auto-injector within tray 104.

In some embodiments, tray 104 may include at least one groove 128. Groove 128 may be located on a sidewall of tray 104. As shown in FIG. 2, cavity 108 may include at least two grooves 128, at least three grooves 128, or at least four grooves 128. Referring to FIGS. 2 and 3A-3C, first sidewall 130a of first plurality of sidewalls 130a, 130b may include at least one groove 128. In other embodiments, second sidewall 130b of first plurality of sidewalls 132a, 132b may include at least one groove 128. In other examples, first sidewall 130a of first plurality of sidewalls 130a, 130b may include at least two grooves 128 and sidewall 130b of first plurality of sidewalls 130a, 130b may include at least two grooves 128. In other embodiments, one or more sidewalls 132a, 132b of second plurality of sidewalls 132a, 132b, may include groove 128. Each groove 128 may have a suitable shape such that device 102 may properly and securely fit into tray 104. As shown in FIGS. 2 and 3A, groove 128 may have a slightly curved shaped. Each groove 128 may be configured to correspond to a portion of retainer 106, which will be discussed in further detail. Each groove 128 may correspond to a portion of retainer 106 to form a snap fit or a friction fit such that retainer 106 may be secured to tray 104 to for packaging 100 for device 102.

As shown in FIG. 2, tray 104 may include at least one nub 134. Nub 134 may be located on one or more sidewalls 130a, 130b of first plurality of sidewalls 130a, 130b. Referring to FIG. 2, at least one nub 134 is located on sidewall 130a and at least one nub 134 is located on sidewall 130b. As shown in FIG. 2, nub 134 may have a generally rectangular shape. Nub 134 may have any appropriate shape and/or configuration in order to secure a portion of medical device 102 within tray 104. For example, nub 134 may form a snap fit with a portion of medical device 102 when medical device 102 is inserted into tray 104.

Referring back to FIGS. 2 and 3A, tray 104 may include a lip 110 extending around at least a portion of tray 104. Lip 110 may be configured to attach to a portion of retainer 106 and/or a portion of removable cover 107 to form a seal to enclose device 102 within packaging 100. In other embodiments, lip 110 may allow for removable, e.g., peelable, cover 107 to be sealed over the top opening of tray 104 to contain device 102 within tray 104 during storage and/or sterilization. An adhesive, e.g., a glue, paste, film, tape, pressure-sensitive material, and/or cold adhesive may be used to seal lip 110 to a portion of retainer 106 and/or to seal a portion of removable cover 107 to lip 110. The adhesive may be compatible for use with the type of external sterilization that may be performed on packaging 100 and/or the sterilant that may be included within packaging 100.

A corner of tray 104, e.g., corner 122 in FIG. 3A, may be free of an adhesive to allow a user to separate a removable cover 107 from tray 104. For example, corner 122 may be free of an adhesive to allow a user to lift a portion of removable cover 107 off of corner 122 in order to remove removable cover 107 from tray 104. As shown in FIGS. 3A, 3B, and 3F, corner 122 may include a tab 124 (or other projection or indentation) to allow a user to separate removable cover 107 from tray 104. For example, tab 124 may be configured such that a user may lift a portion of removable cover off of corner 122 in order to remove removable cover 107 from tray 104. Tab 124 may be correspond to a portion of, e.g., a corner of, removable cover 107, which will be discussed in further detail.

FIG. 3B depicts a cross-section view corresponding to section A-A of FIG. 3A. In some embodiments, a length of tray 104 may range from about 4 inches to about 7 inches. For example, a length of tray 104 may range from about 4 inches to about 6.5 inches, about 4 inches to about 6 inches, about 4 inches to about 5.5 inches, about 4.5 inches to about 7 inches, about 4.5 inches to about 6.5 inches, about 4.5 inches to about 6 inches, about 4.5 inches to about 5.5 inches, about 5 inches to about 7 inches, about 5 inches to about 6.5 inches, or about 5 inches to about 6 inches. In some examples, a length of tray 104 may range from about 4.5 inches to about 6.3 inches, about 4.5 inches to about 6.1 inches, about 4.5 inches to about 5.9 inches, about 4.5 inches to about 5.7 inches, about 4.5 inches to about 5.5 inches, about 4.5 inches to about 5.4 inches, about 4.7 inches to about 5.4 inches, about 4.9 inches to about 5.4 inches, about 5.1 inches to about 5.4 inches. In some examples, a length of tray 104 may be about 5 inches, about 5.1 inches, about 5.2 inches, about 5.3 inches, about 5.4 inches, about 5.5 inches, about 5.6 inches, about 5.7 inches, about 5.8 inches, or about 5.9 inches.

Referring to FIG. 3A, each edge of tray 104 may have a radius of curvature R ranging from about 30 R to 50 R. For example, the radius of curvature R may range from about 30 R to about 45 R, about 35 R to about 45 R, about 35 R to about 50 R, about 40 R to about 45 R, about 40 R to about 50 R, about 42 R to about 50 R, about 42 R to about 48 R, or about 42 R to about 46 R. In some examples, the radius of curvature R may be about 30 R, 31, R, 32, R, 33 R, 34 R, 35 R, 36 R, 37 R, 38 R, 39 R, 40 R, 41 R, 42 R, 43 R, 44 R, 45 R, 46 R, 47 R, 48 R, 49 R, or 50 R.

FIG. 3C depicts a cross-section view corresponding to section B-B of FIG. 3A. In some embodiments, a width of tray 104 may range from about 3 inches to about 6 inches. For example, a width of tray 104 may range from about 3 inches to about 5.5 inches, about 3 inches to about 5 inches, about 3.5 inches to about 6 inches, about 3.5 inches to about 5.5 inches, about 3.5 inches to about 5 inches, about 4 inches to about 6 inches, about 4 inches to about 5.5 inches, about 4 inches to about 5 inches, about 4.5 inches to about 6 inches, about 4.5 inches to about 5.5 inches, about 4.5 inches to about 5 inches. In some examples, a width of tray 104 may be about 4 inches, about 4.1 inches, about 4.2 inches, about 4.3 inches, about 4.4 inches, about 4.5 inches, about 4.6 inches, about 4.7 inches, about 4.8 inches, or about 4.9 inches.

Referring to FIG. 3C, a height H of tray 104 may range from about 1 inch to about 2 inches. For example, a height H of tray 104 may range from about 1 inch to about 1.9 inches, about 1 inch to about 1.8 inches, about 1 inch to about 1.7 inches, about 1 inch to about 1.6 inches, about 1 inch to about 1.5 inches, about 1 inch to about 1.4 inches, about 1 inch to about 1.3 inches, about 1.1 inches to about 2 inches, about 1.2 inches to about 2 inches, about 1.1 inches to about 1.9 inches, about 1.1 inches to about 1.8 inches, about 1.1 inches to about 1.7 inches, about 1.1 inches to about 1.6 inches, about 1.1 inches to about 1.5 inches, about 1.1 inches to about 1.4 inches, about 1.2 inches to about 2 inches, about 1.2 inches to about 1.9 inches, about 1.2 inches to about 1.8 inches, about 1.2 inches to about 1.7 inches, about 1.2 inches to about 1.6 inches, about 1.2 inches to about 1.5 inches, or about 1.2 inches to about 1.4 inches.

As shown in FIG. 3C, a width I of cavity 108 may range from about 2 inches to about 4 inches. For example, a width I of cavity may range from about 2 inches to about 3.9 inches, about 2 inches to about 3.8 inches, about 2 inches to about 3.7 inches, about 2 inches to about 3.6 inches, about 2 inches to about 3.5 inches, about 2 inches to about 3.4 inches, about 2 inches to about 3.3 inches, about 2 inches to about 3.2 inches, about 2 inches to about 3.1 inches, about 2 inches to about 3 inches, about 2.1 inches to about 3 inches, about 2.2 inches to about 3 inches, about 2.3 inches to about 3 inches, about 2.4 inches to about 3 inches, about 2.5 inches to about 3 inches, about 2.6 inches to about 3 inches, about 2.7 inches to about 3 inches, about 2.8 inches to about 3 inches, about 2.9 inches to about 3 inches, about 2.5 inches to about 4 inches, about 2.6 inches to about 4 inches, about 2.7 inches to about 4 inches, about 2.8 inches to about 4 inches, or about 2.9 inches to about 4 inches.

Also referring to FIG. 3C, a width J of lip 110 may range from about 0.1 inch to about 0.5 inch. For example, a width J of lip 110 may range from about 0.1 inch to about 0.45 inch, about 0.1 inch to about 0.4 inch, about 0.1 inch to about 0.35 inch, about 0.15 inch to about 0.5 inch, about 0.15 inch to about 0.45 inch, about 0.15 inch to about 0.4 inch, about 0.15 inch to about 0.3 inch, about 0.2 inch to about 0.5 inch, about 0.2 inch to about 0.45 inch, about 0.2 inch to about 0.4 inch, about 0.2 inch to about 0.35 inch, about 0.2 inch to about 0.3 inch, about 0.25 inch to about 0.3 inch, about 0.25 inch to about 0.35 inch, about 0.25 inch to about 0.4 inch, about 0.25 inch to about 0.5 inch.

FIG. 3D depicts a cross-section view corresponding to section C-C of FIG. 3A. As shown in FIG. 3D, a portion of tab 124 may extend above lip 110 at a distance K ranging from about 0.01 inch to about 0.5 inch. For example, distance K may range from about 0.01 inch to about 0.4 inch, about 0.01 inch to about 0.3 inch, about 0.01 inch to about 0.2 inch, about 0.01 inch to about 0.1 inch, about 0.01 inch to about 0.09 inch, about 0.01 inch to about 0.08 inch, about 0.01 inch to about 0.07 inch, about 0.01 inch to about 0.06 inch, about 0.01 inch to about 0.05 inch, about 0.01 to about 0.04 inch, about 0.02 inch to about 0.5 inch, about 0.02 inch to about 0.4 inch, about 0.02 inch to about 0.3 inch, about 0.02 inch to about 0.2 inch, about 0.02 inch to about 0.1 inch, about 0.02 inch to about 0.09 inch, about 0.02 inch to about 0.08 inch about 0.02 inch to about 0.07 inch, about 0.02 inch to about 0.06 inch, about 0.02 inch to about 0.05 inch, or about 0.02 inch to about 0.04 inch.

Also referring to FIG. 3D, the outer edge of corner 122 to the outer edge of lip 110 corresponds to distance L. Distance L may range from about 0.5 inch to about 1.5 inches. For example, distance L may range from about 0.5 inch to about 1.4 inches, about 0.5 inch to about 1.3 inches, about 0.5 inch to about 1.2 inches, about 0.5 inch to about 1.1 inches, about 0.5 inch to about 1 inch, about 0.5 inch to about 0.9 inch, about 0.6 inch to about 1.5 inches, about 0.6 inch to about 1.4 inches, about 0.6 inch to about 1.3 inches, about 0.6 inch to about 1.2 inches, about 0.6 inch to about 1.1 inches, about 0.6 inch to about 1 inch, about 0.6 inch to about 0.9 inch, about 0.7 inch to about 1.5 inches, about 0.7 inch to about 1.4 inches, about 0.7 inch to about 1.3 inches, about 0.7 inch to about 1.2 inches, about 0.7 inch to about 1.1 inches, about 0.7 inch to about 1 inch, about 0.7 inch to about 0.9 inch, about 0.8 inch to about 1.5 inches, about 0.8 inch to about 1.4 inches, about 0.8 inch to about 1.3 inches, about 0.8 inch to about 1.3 inches, about 0.8 inch to about 1.2 inches, about 0.8 inch to about 1.1 inches, or about 0.8 inch to about 1 inch.

FIG. 3E depicts an enlarged view of detail D of FIG. 3B. As shown in FIG. 3E, a height E of lip 110 on an outer edge of tray 104 may range from about 0.05 inch to 0.5 inch. For example, a height E of lip 110 may range from about 0.05 inch to about 0.4 inch, about 0.05 inch to about 0.3 inch, about 0.05 inch to about 0.2 inch, about 0.05 inch to about 0.1 inch, about 0.06 inch to about 0.5 inch, about 0.06 inch to about 0.4 inch, about 0.06 inch to about 0.3 inch, about 0.06 inch to about 0.2 inch, about 0.06 inch to about 0.1 inch, about 0.07 inch to about 0.5 inch, about 0.07 inch to about 0.4 inch, about 0.07 inch to about 0.3 inch, about 0.07 inch to about 0.2 inch, about 0.07 inch to about 0.1 inch, or about 0.07 inch to about 0.09 inch.

FIG. 3F depicts a bottom view of tray 104. FIG. 3G depicts a cross-section view corresponding to section F-F of FIG. 3F and FIG. 3H depicts a cross-section view corresponding to section G-G of FIG. 3F. A width M of the external surface of cavity 108, as depicted in FIG. 3G, may range from about 3 inches to about 4 inches. For example, width M may range from about 3 inches to about 3.9 inches, about 3 inches to about 3.8 inches, about 3 inches to about 3.7 inches, about 3 inches to about 3.6 inches, about 3 inches to about 3.5 inches, about 3 inches to about 3.4 inches, about 3 inches to about 3.3 inches, or about 3 inches to about 3.2 inches. A width N of the external surface of cavity 108, as depicted in FIG. 3H, may range from about 3 inches to about 4 inches. For example, width N may range from about 3 inches to about 3.9 inches, about 3 inches to about 3.8 inches, about 3 inches to about 3.7 inches, about 3 inches to about 3.6 inches, about 3 inches to about 3.5 inches, about 3 inches to about 3.4 inches, about 3 inches to about 3.3 inches, or about 3 inches to about 3.2 inches.

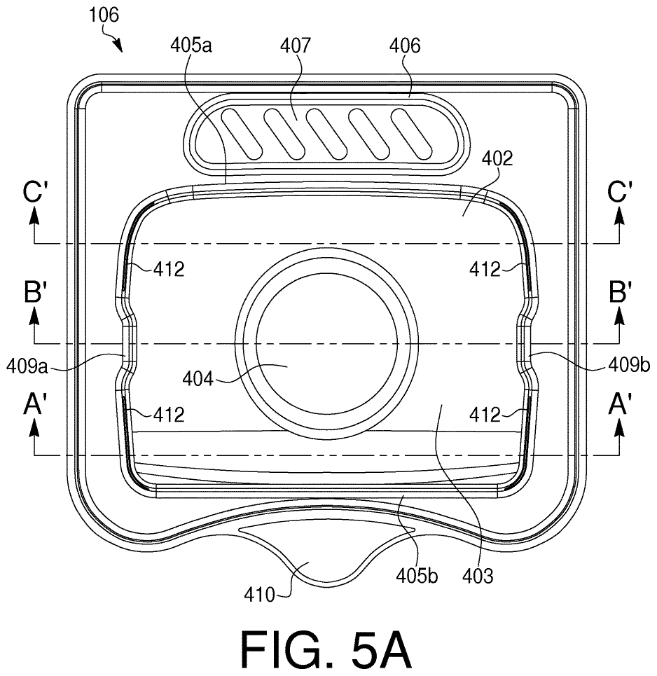

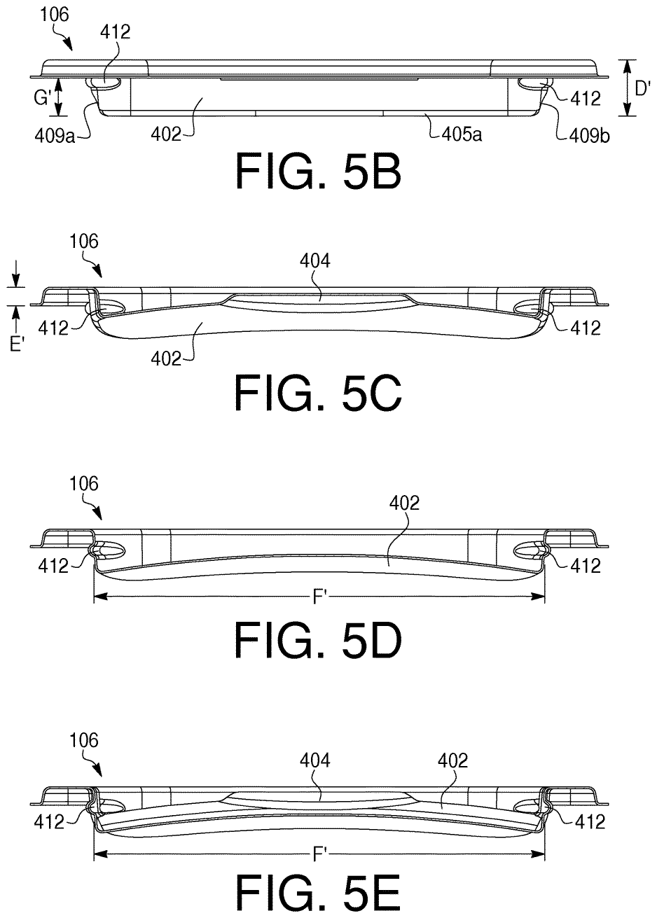

FIGS. 4-5E depict an exemplary retainer 106 of packaging 100. Referring to FIG. 4, retainer 106 may include a plurality of cavities 402, 406. Retainer 106 may have a first cavity 402 and a second cavity 406. In other embodiments, retainer 106 may include at least two cavities, at least three cavities, or at least four cavities. As shown in FIG. 4, first cavity 402 may include a base 403, a first plurality of sidewalls 405a, 405b, and a second plurality of sidewalls 409a, 409b. In some embodiments, a length of first plurality of sidewalls 405a, 405b may be larger than a length of second plurality of sidewalls 409a, 409b. In other embodiments, a length of first plurality of sidewalls 405a, 405b may be less than a length of second plurality of sidewalls 409a, 409b. In other embodiments, a length of first plurality of sidewalls 405a, 405b may be equal to a length of second plurality of sidewalls 409a, 409b. In other embodiments, second cavity 406 may include a base 407.

As shown in FIG. 4, retainer 106 may include a dome 404 and a plurality of apertures 412. In some embodiments, base 403 of retainer 106 may include dome 404. Dome 404 may have a suitable shape such that dome 404 abuts to and/or forms a friction fit with a portion of device 102, e.g., as shown in FIG. 1, when retainer 106 is attached to tray 104 to form packaging 100. Dome 404 may protect a portion of device 102, e.g., an upper portion of device 102, and/or interface with a portion of device 102. In some embodiments, dome 404 may contain a sterilant as discussed above.

Referring to FIGS. 4 and 5A, retainer 106 may include at least two apertures 412, at least three apertures 412, or at least four apertures 412. Each aperture 412 may have a suitable shape such that when device 102 is placed into cavity 108 of tray 104 and retainer 106 is sealed to a portion of tray 104, retainer 106 may securely and properly fit with and/or seal with tray 104 and each aperture 412 may fit into a corresponding groove 128 of tray 104, as shown in FIGS. 2 and 3A. As shown in FIG. 4, aperture 412 may have a slightly curved shape. Each aperture 412 may be configured to correspond to the shape of corresponding groove 128 to form a friction fit such that retainer 106 may be secured to tray 104 for packaging 100 for device 102. Each aperture 412 may also be placed at a location of first cavity 402 such that each aperture 412 properly secures to and/or is received in corresponding groove 128. For example, each sidewall of first plurality of sidewalls 405a, 405b may include a portion of aperture 412. In other embodiments, each sidewall of second plurality of sidewalls 409a, 409b may include a portion of aperture 412. As shown in FIGS. 4 and 5A, aperture 412 may have a slightly curved shaped such that at least one aperture 412 may extend from a portion of sidewall 405a to a portion of sidewall 409a, at least one aperture 412 may extend from a portion of sidewall 405a to a portion of sidewall 409b, at least one aperture 412 may extend from a portion of sidewall 405b to a portion of sidewall 409a, and at least one aperture 412 may extend from a portion of sidewall 405b to a portion of sidewall 409b.

Second cavity 406 of retainer 106 may include a base 407. In some embodiments, base 407 may include at least one vent 408. When retainer 106 is secured to tray 104 to enclose device 102, vent 408 may allow for airflow into tray 104 and/or around device 102. Vent 408 may also allow for sterilization and/or for any sterilant contained in packaging 100, e.g., in tray 104, to flow within packaging 100. Vent 408 may encourage the flow of and/or increase the penetration of sterilant gas into packaging 100 and its components. Base 407 of second cavity 406 may include a plurality of vents 408, e.g., at least one vent 408, at least two vents 408, at least three vents 408, at least four vents 408, or at least five vents, etc. As shown in FIG. 4, first cavity 402 has a generally square shape and second cavity 406 has a generally rectangular shape. First cavity 402 and second cavity 406 may have any appropriate shape or configuration that allows retainer 106 to properly secure to, abut against, and/or form a friction fit with, tray 104. Retainer 106 may also include an overhang 410 for assisting a user in separating retainer 106 from tray 104, e.g., the removal of retainer 106 from tray 104. Overhang 410 may have any appropriate shape allowing retainer 106 to properly secure to, abut against, and/or form a friction fit with, tray 104, and allowing for a user to properly separate retainer 106 from tray 104. During use, after a user removes removable cover 107 from tray 104, a user may grasp a portion of overhang 410 and lift a portion of overhang 410 in an upwards motion to separate retainer 106 from tray 104. The user may then remove the device from tray 104.

Retainer 106 may be made of a compressible material that deforms under pressure, e.g., pressure applied by a user. In some embodiments, retainer 106 may be thermoformed plastic. For example, retainer 106 may be formed of one or more of polypropylene, polystyrene, polyethylene, polycarbonate, polyvinyl chloride, polyethylene terephthalate glycol (PETG).

Referring to FIG. 5A, a length of retainer 106 extending from a top edge of retainer 106 to a bottom edge of overhang 410 may range from about 3 inches to about 4 inches. For example, a length of retainer 106 may range from about 3 inches to about 3.9 inches, about 3 inches to about 3.8 inches, about 3.1 inches to about 4 inches, about 3.2 inches to about 4 inches, about 3.3 inches to about 4 inches, about 3.4 inches to about 4 inches, about 3.5 inches to about 4 inches, about 3.6 inches to about 4 inches, about 3.7 inches to about 4 inches, about 3.7 inches to about 3.8 inches, about 3.7 inches to about 3.9 inches, about 3.6 inches to about 3.8 inches, about 3.6 inches to about 3.9 inches, about 3.5 inches to about 3.8 inches, or about 3.5 inches to about 3.9 inches.

A width of retainer 106 may range from about 3 inches to about 4 inches. For example, a width of retainer 106 may range from about 3.1 inches to about 4 inches, about 3.2 inches to about 4 inches, about 3.3 inches to about 4 inches, about 3.4 inches to about 4 inches, about 3.5 inches to about 4 inches, about 3.6 inches to about 4 inches, about 3.7 inches to about 4 inches, about 3.8 inches to about 4 inches, about 3 inches to about 3.9 inches, about 3.1 inches to about 3.9 inches, about 3.2 inches to about 3.9 inches, about 3.3 inches to about 3.9 inches, about 3.4 inches to about 3.9 inches, about 3.5 inches to about 3.9 inches, about 3.6 inches to about 3.9 inches, about 3.7 inches to about 3.9 inches, or about 3.8 inches to about 3.9 inches.

FIG. 5B depicts a front view of retainer 106. A height D′ of retainer 106 may range from about 0.2 inches to about 0.5 inches. For example, height D′ of retainer 106 may range from about 0.2 inches to about 0.49 inches, about 0.2 inches to about 0.45 inches, about 0.2 inches to about 0.43 inches, about 0.2 inches to about 0.4 inches, about 0.23 inches to about 0.5 inches, about 0.25 inches to about 0.5 inches, about 0.27 inches to about 0.5 inches, about 0.3 inches to about 0.5 inches, about 0.33 inches to about 0.5 inches, about 0.35 inches to about 0.5 inches, about 0.3 inches to about 0.45 inches, about 0.3 inches to about 0.43 inches, about 0.3 inches to about 0.4 inches, or about 0.35 inches to about 0.4 inches.

Referring to FIG. 5B, any one of first plurality of sidewalls 405a, 405b, second plurality of sidewalls 409a, 409b, and base 403 may extend away from a surface of retainer 106 such that when retainer 106 is secured to tray 104 to enclose device 102, at least a portion of base 403 abuts against a portion of device 102. A height G′ of first cavity 402 may range from about 0.1 inches to about 0.4 inches. For example, height G′ of first cavity 402 may range from about 0.15 inches to about 0.4 inches, about 0.18 inches to about 0.4 inches, about 0.2 inches to about 0.4 inches, about 0.22 inches to about 0.4 inches, about 0.1 inches to about 0.35 inches, about 0.1 inches to about 0.33 inches, about 0.1 inches to about 0.3 inches, about 0.1 inches to about 0.28 inches, about 0.15 inches to about 0.28 inches, about 0.15 inches to about 0.3 inches, about 0.15 inches to about 0.33 inches, about 0.15 inches to about 0.35 inches, about 0.18 inches to about 0.3 inches, about 0.18 inches to about 0.28 inches, about 0.2 inches to about 0.28 inches, about 0.2 inches to about 0.3 inches, about 0.22 inches to about 0.3 inches, about 0.22 inches to about 0.28 inches, about 0.22 inches to about 0.32 inches, about 0.2 inches to about 0.29 inches, or about 0.21 inches to about 0.29 inches.

FIG. 5C depicts a cross-section view corresponding to section B′-B′ of FIG. 5A. Referring to FIG. 5C, a height E′ of the portion of retainer 106 surrounding first cavity 402 may range from about 0.01 inches to about 0.25 inches. This portion of retainer 106 may be referred to as a ledge. For example, height E′ of the ledge of retainer 106 may range from about 0.01 inches to about 0.22 inches, about 0.01 inches to about 0.2 inches, about 0.01 inches to about 0.18 inches, about 0.01 inches to about 0.16 inches, about 0.01 inches to about 0.14 inches, about 0.05 inches to about 0.25 inches, about 0.05 inches to about 0.22 inches, about 0.05 inches to about 0.2 inches, about 0.05 inches to about 0.18 inches, about 0.05 inches to about 0.15 inches, about 0.05 inches to about 0.13 inches, 0.08 inches to about 0.13 inches, about 0.1 inches to about 0.13 inches, about 0.1 inches to about 0.25 inches, about 0.1 inches to about 0.22 inches, about 0.1 inches to about 0.2 inches, about 0.1 inches to about 0.18 inches, or about 0.1 inches to about 0.15 inches.

FIG. 5D depicts a cross-section view corresponding to section C′-C′ OF FIG. 5A and FIG. 5E depicts a cross-section view corresponding to section A′-A′ of FIG. 5A. Referring to FIGS. 5D and 5E, a width F′ of cavity 402 may range from about 2.5 inches to about 4.5 inches. For example, width F′ of cavity 402 may range from about 2.6 inches to about 4.5 inches, about 2.7 inches to about 4.5 inches, about 2.8 inches to about 4.5 inches, about 2.9 inches to about 4.5 inches, about 3 inches to about 4.5 inches, about 3 inches to about 4.4 inches, about 3 inches to about 4.3 inches, about 3 inches to about 4.2 inches, about 3 inches to about 4.1 inches, about 3 inches to about 4 inches, about 3 inches to about 3.9 inches, about 3 inches to about 3.8 inches, about 3 inches to about 3.7 inches, about 3 inches to about 3.6 inches, about 3 inches to about 3.5 inches, about 3 inches to about 3.4 inches, about 3 inches to about 3.3 inches, about 3 inches to about 3.2 inches, about 3 inches to about 3.1 inches, about 2.9 inches to about 3.1 inches, or about 2.8 inches to about 3.1 inches.

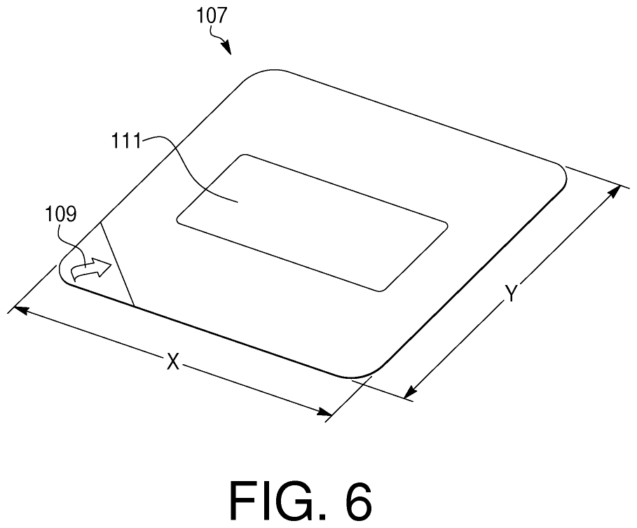

FIG. 6 depicts removable cover 107. Removable cover 107 may be permeable to vapor hydrogen peroxide and/or an ethyl alcohol. Removable cover 107 may be formed of, e.g., Tyvek or other suitable high-density polyethylene fibers, ethylene-vinyl acetate, and/or other thermoplastic materials. Removable cover 107 may be permeable to a suitable gaseous sterilant, such as ethylene oxide, vaporized hydrogen peroxide, ethyl alcohol, nitrous oxide, nitrogen dioxide, or any appropriate sterilant in the art. As mentioned above, components of packaging 100, e.g., tray 104, retainer 106, and/or removable cover 107, and the size and/or shape of such components may be configured to facilitate high speed manufacturing of packaging 100 and/or improve material distribution of packaging 100.

As shown in FIG. 6, removable cover 107 may include a flap 109. Flap 109 may be configured such that a user may grasp a portion of flap 109 to lift a portion of removable cover 107 off of corner 122 and/or tab 124 of tray 104 in order to remove removable cover 107 from tray 104. Removable cover 107 may include an adhesive, e.g., a glue, paste, film, tape, pressure-sensitive material, and/or cold adhesive. For example, such adhesive may be applied to a portion of removable cover 107 that adheres to tray 104. The adhesive of removable cover 107 may be used to seal a portion of removable cover 107 to a portion of tray 104, e.g., lip 110 of tray 104. The adhesive may be compatible for use with the type of external sterilization that may be performed on packaging 100 and/or the sterilant that may be included within packaging 100. In some examples, flap 109 of removable cover 107 may be free of any adhesive. In packaging 100, removable cover 107 may not adhere to and/or secure any portion of medical device 102. Removable cover 107 is secured to a portion of tray 104 in order to form an enclosure for medical device 102 and/or retainer 106. Such enclosure formed by removable cover 107 and tray 104 allows for proper storage and/or sterilization of medical device 102.

Referring to FIG. 6, a length Y of removable cover 107 may range from about 4 inches to about 7 inches. For example, a length Y of removable cover 107 may range from about 4 inches to about 6.5 inches, about 4 inches to about 6 inches, about 4 inches to about 5.5 inches, about 4.5 inches to about 7 inches, about 4.5 inches to about 6.5 inches, about 4.5 inches to about 6 inches, about 4.5 inches to about 5.5 inches, about 5 inches to about 7 inches, about 5 inches to about 6.5 inches, or about 5 inches to about 6 inches. In some examples, a length Y of removable cover 7 may range from about 4.5 inches to about 6.3 inches, about 4.5 inches to about 6.1 inches, about 4.5 inches to about 5.9 inches, about 4.5 inches to about 5.7 inches, about 4.5 inches to about 5.5 inches, about 4.5 inches to about 5.4 inches, about 4.7 inches to about 5.4 inches, about 4.9 inches to about 5.4 inches, about 5.1 inches to about 5.4 inches. In some examples, a length Y of removable cover 107 may be about 5 inches, about 5.1 inches, about 5.2 inches, about 5.3 inches, about 5.4 inches, about 5.5 inches, about 5.6 inches, about 5.7 inches, about 5.8 inches, or about 5.9 inches.

Referring to FIG. 6, a width X of removable cover 107 may range from about 3 inches to about 6 inches. For example, a width X of removable cover 107 may range from about 3 inches to about 5.5 inches, about 3 inches to about 5 inches, about 3.5 inches to about 6 inches, about 3.5 inches to about 5.5 inches, about 3.5 inches to about 5 inches, about 4 inches to about 6 inches, about 4 inches to about 5.5 inches, about 4 inches to about 5 inches, about 4.5 inches to about 6 inches, about 4.5 inches to about 5.5 inches, about 4.5 inches to about 5 inches. In some examples, a width X of removable cover 107 may be about 4 inches, about 4.1 inches, about 4.2 inches, about 4.3 inches, about 4.4 inches, about 4.5 inches, about 4.6 inches, about 4.7 inches, about 4.8 inches, or about 4.9 inches.

Exemplary medicaments, drugs, and/or pharmaceutical formulations that may be used with the medical devices of the disclosure, e.g., auto-injectors, include: pharmaceuticals targeting Activin A and GDF8 (e.g., garetosmab and trevogrumab as described in U.S. Pat. No. 9,718,881, which is incorporated by reference herein); pharmaceuticals targeting C5 (e.g., pozelimab and cemdisiran, as described in US Publication 2021/0046182, which is incorporated by reference herein); pharmaceuticals targeting LEPR (e.g., mibavademab, as described in U.S. Pat. No. 10,023,644, which is incorporated by reference herein); pharmaceuticals targeting LAG3 (e.g., fianlimab, as described in U.S. Pat. No. 10,358,495, which is incorporated by reference herein); pharmaceuticals targeting BetV1 (e.g., antibodies disclosed in U.S. Pat. No. 10,793,624, which is incorporated by reference herein); pharmaceuticals targeting PCSK9 (e.g., alirocumab, as described in U.S. Pat. No. 8,795,669, which is incorporated by reference herein); pharmaceuticals targeting ANGPTL3 (e.g., evinacumab, as disclosed in US Publication 2020/0369760, which is incorporated by reference herein); pharmaceuticals targeting Ebola (e.g., atolivimab, maftivimab, odesivimab, as disclosed in US Publication 2021/0252146, which is incorporated by reference herein); pharmaceuticals targeting IL-6R (e.g., sarilumab, as disclosed in U.S. Pat. No. 9,173,880, which is incorporated by reference herein).

Additional exemplary medicaments, drugs, and/or pharmaceutical formulations include: RNAi therapeutic targeting APP for early-onset Alzheimer's disease (ALN-APP1); RNAi therapeutic targeting HSD17B13 for nonalcoholic steatohepatitis (“NASH”) (ALN-HSD); RNAi therapeutic targeting PNPLA3 for NASH (ALN-PNP1); PD-1 Antibody for First-line NSCLC, BNT116 combination (CEMIPLIMAB); Bispecific antibody targeting BCMA and CD3 for Multiple myeloma (LINVOSELTAMAB); TTR gene knockout using CRISPR/Cas9 for Transthyretin (“ATTR”) amyloidosis; Bispecific antibody targeting CD20 and CD3 for certain B-cell malignancies (ODRONEXTAMAB); Bispecific antibody targeting PSMA and CD3 for prostate cancer; Bispecific antibody targeting two distinct MET epitopes for MET-altered advanced NSCLC; Bispecific antibody-drug conjugate targeting two distinct MET epitopes for MET overexpressing advanced cancer; Agonist Antibody to NPR1/Reversal Agent to REGN5381 for reversal agent in healthy volunteers; Bispecific antibody targeting BCMA and CD3 for transplant desensitization in patients with chronic kidney disease; Bispecific antibody targeting MUC16 and CD28 for platinum-resistant ovarian cancer; Bispecific antibody targeting PSMA and CD28 for prostate cancer; Bispecific antibody targeting CD22 and CD28 for B-NHL; Antibody to GITR for solid tumors; Bispecific antibody targeting EGFR and CD28 for solid tumors; Antibody to IL2Rg for aplastic anemia; Antibody to Factor XI for thrombosis; Antibody to TMPRSS6 for transfusion dependent iron overload; Antibody to Factor XI for thrombosis; RNAi therapeutic targeting HSD17B13 for nonalcoholic steatohepatitis (“NASH”) (ALN-HSD); Antibody to PD-1 Neoadjuvant CSCC; for second-line cervical cancer, ISA101b combination (CEMIPLIMAB); Antibody to IL-4R alpha subunit for ulcerative colitis; Eosinophilic gastroenteritis (Phase 2/3) (DUPILUMAB); Antibody to LAG-3 for first-line advanced NSCLC (Phase 2/3) (pivotal study) (FIANLIMAB); Bispecific antibody targeting BCMA and CD3 for multiple myeloma (pivotal study) (LINVOSELTAMAB); Agonist antibody to leptin receptor (“LEPR”) for generalized lipodystrophy; Partial lipodystrophy (MIBAVADEMAB); Bispecific antibody targeting CD20 and CD3 for B-cell non-Hodgkin lymphoma (“B-NHL”) (pivotal study) (ODRONEXTAMAB); Antibody to C5; studied as monotherapy and in combination with cemdisiran for CD55-deficient protein-losing enteropathy (“CHAPLE”), monotherapy (potentially pivotal study) (POZELIMAB); Agonist Antibody to NPR1/Reversal Agent to REGN5381 for heart failure; Antibody to IL-6R for polyarticular-course juvenile idiopathic arthritis (“pcJIA”) (pivotal study); systemic juvenile idiopathic arthritis (“sJIA”) (pivotal study) (SARILUMAB); Bispecific antibody targeting MUC16 and CD3 for platinum-resistant ovarian cancer (UBAMATAMAB); Immune activator targeting TLR9 for solid tumors (VIDUTOLIMOD); VEGF-Trap for Wet AMD, DME (AFLIBERCEPT); Antibody to PCSK9 for HeFH in pediatrics (ALIROCUMAB); Antibody to PD-1 for adjuvant CSCC (CEMIPLIMAB); Antibody to IL-4R alpha subunit for EoE in pediatrics; chronic obstructive pulmonary disease (“COPD”); bullous pemphigoid; chronic spontaneous urticaria (“CSU”); chronic pruritis of unknown origin (DUPILUMAB); Antibody to LAG-3 for first-line metastatic melanoma; First-line adjuvant melanoma (FIANLIMAB); Antibody to Activin A for fibrodysplasia ossificans progressiva (“FOP”) (GARETOSMAB); Antibody to IL-33 for COPD (ITEPEKIMAB); Antibody to C5; studied as monotherapy and in combination with cemdisiran for myasthenia gravis, cemdisiran combination; paroxysmal nocturnal hemoglobinuria (“PNH”), cemdisiran combination (POZELIMAB); Multi-antibody therapy to Bet v 1 for birch allergy.

The description above and examples are illustrative, and are not intended to be restrictive. One of ordinary skill in the art may make numerous modifications and/or changes without departing from the general scope of the invention. For example, and as has been referenced, the above-described embodiments (and/or aspects thereof) may be used in combination with each other. Additionally, portions of the above-described embodiments may be removed without departing from the scope of the invention. In addition, modifications may be made to adapt a particular situation or aspect to the teachings of the various embodiments without departing from their scope. Many other embodiments will also be apparent to those of skill in the art upon reviewing the above description. Moreover, the features of any embodiment may be used in conjunction with any of the other disclosed embodiments.

Embodiments of the present disclosure may include the following features:

Item 1. A packaging comprising:
a tray having a body surrounding an exposed cavity and a groove;
a lip surrounding the opening, wherein the lip extends radially outward from the cavity and defines a periphery of the tray;
a retainer for covering a portion of the opening of the tray, the retainer having an aperture, wherein the aperture corresponds to the groove of the tray;
a removable cover; and
an auto-injector contained within the cavity, wherein the auto-injector is pre-filed with a medicament or other fluid.

Item 2. The packaging of item 1, further comprising a gaseous sterilant.

Item 3. The packaging of item 2, wherein the gaseous sterilant includes one or more of ethylene oxide, vaporized hydrogen peroxide, ethyl alcohol, nitrous oxide, or nitrogen dioxide.

Item 4. The packaging of item 1, wherein the tray includes a first plurality of sidewalls and a second plurality of sidewalls.

Item 5. The packaging of item 1, further comprising a lug.

Item 6. The packaging of item 1, wherein the lip includes an adhesive and forms a seal with the retainer to enclose the auto-injector within the cavity.

Item 7. The packaging of item 1, wherein the tray and/or the retainer includes one or more of polypropylene, polystyrene, polyethylene, polycarbonate, polyvinyl chloride, or polyethylene terephthalate glycol.

Item 8. The packaging of item 1, wherein the removable cover includes high-density polyethylene fibers, thermoplastic materials, or a combination thereof.

Item 9. The packaging of item 1, wherein the medicament or fluid comprises one or more of dupilumab and cemiplimab.

Item 10. A packaging for an auto-injector, the packaging comprising:
a tray having an opening and a cavity;
a lip surrounding the opening, wherein the lip extends radially outward from the cavity and defines a periphery of the tray;
a removable cover having a periphery that is adhered to the lip; and
a retainer for covering a portion of the opening of the tray.

Item 11. The packaging of item 10, wherein the removable cover is permeable to a gaseous sterilant.

Item 12. The packaging of item 10, wherein the gaseous sterilant includes one or more of ethylene oxide, vaporized hydrogen peroxide, ethyl alcohol, nitrous oxide, or nitrogen dioxide.

Item 13. The packaging of item 10, wherein the tray and/or the retainer includes one or more of polypropylene, polystyrene, polyethylene, polycarbonate, polyvinyl chloride, or polyethylene terephthalate glycol.

Item 14. The packaging of item 10, wherein the removable cover includes high-density polyethylene fibers, thermoplastic materials, or a combination thereof.

Item 15. The packaging of item 10, wherein the auto-injector contains a medicament comprising one or more of dupilumab and cemiplimab.

Item 16. A method of removing an externally-sterilized auto-injector from a packaging, the packaging including a tray having an opening and a cavity, a retainer, a removable cover, and a gaseous sterilant, the method comprising:
removing the removable cover to expose the retainer;
removing the retainer to expose the cavity of the tray;
grasping a portion of the externally-sterilized auto-injector held within the cavity of the tray; and
ejecting the externally-sterilized auto-injector from the tray.

Item 17. The method of item 16, further comprising pushing a portion of the tray towards the cavity of the tray, wherein pushing the portion of the tray causes a portion of the tray to flex.

Item 18. The method of item 17, wherein pushing the portion of the tray causes the externally-sterilized auto-injector to release from the cavity of the tray.

Item 19. The method of item 18, wherein the gaseous sterilant includes one or more of ethylene oxide, vaporized hydrogen peroxide, ethyl alcohol, nitrous oxide, or nitrogen dioxide.

Item 20. The method of item 19, wherein the externally-sterilized auto-injector contains a medicament comprising one or more of dupilumab and cemiplimab.

What is claimed is:

1. A packaging, comprising:
a tray including a first sidewall and an exposed cavity defined in part by the first sidewall, wherein the first sidewall includes a groove, and wherein the exposed cavity is shaped to receive a medical device;
a lip extending along an upper portion of the tray; and
a retainer including a second sidewall, wherein the second sidewall includes a protrusion, wherein the retainer is configured to fit over the medical device within the exposed cavity, and wherein the protrusion is configured to form a friction fit with the groove.

2. The packaging of claim 1, wherein the tray further includes a third sidewall opposite the first sidewall, and wherein the third sidewall includes a second groove.

3. The packaging of claim 2, wherein the retainer includes a fourth sidewall opposite the second sidewall, and wherein the fourth sidewall includes a second protrusion configured to form a friction fit with the second groove.

4. The packaging of claim 1, wherein the protrusion is configured to snap into the groove when the retainer is positioned over the medical device and the tray.

5. The packaging of claim 1, wherein the tray further includes a lug shaped to form a space between a portion of the medical device and the tray when the medical device is received in the exposed cavity.

6. The packaging of claim 5, wherein the lug is one of four lugs.

7. The packaging of claim 1, wherein the tray further includes a depression on a bottom portion of the tray, and wherein the depression is shaped to receive a lock of the medical device.

8. The packaging of claim 1, wherein the tray further includes a nub located on the first sidewall, and wherein the nub is configured to form a snap fit with a portion of the medical device when the medical device is inserted into the tray.

9. The packaging of claim 1, wherein the retainer includes a first cavity and a second cavity, wherein the first cavity includes a dome configured to abut a portion of the medical device when the retainer is attached to the tray.

10. The packaging of claim 9, wherein the second cavity includes a base having at least one vent configured to allow airflow into the tray.

11. A method for packaging a medical device, the method comprising:
positioning the medical device within an exposed cavity of a tray, wherein the tray includes a first sidewall having a groove, wherein the exposed cavity is defined in part by the first sidewall, and wherein the exposed cavity is shaped to receive the medical device; and
positioning a retainer including a second sidewall over the medical device and the tray, wherein the second sidewall includes a protrusion, and wherein the protrusion is configured to form a friction fit with the groove.

12. The method of claim 11, wherein positioning the retainer over the medical device and the tray causes the protrusion to snap into the groove.

13. The method of claim 11, wherein, during the positioning of the retainer, the friction fit between the protrusion and the groove secures the retainer to the tray to enclose the medical device within the exposed cavity.

14. The method of claim 11, further comprising adhering a removable cover to a lip of the tray, wherein the lip extends along an upper portion of the tray.

15. The method of claim 14, wherein adhering the removable cover to the lip forms a seal that encloses the retainer and the medical device within the tray.

16. The method of claim 15, further comprising sterilizing the medical device with a gaseous sterilant after adhering the removable cover to the lip.

17. The method of claim 16, wherein the removable cover is permeable to the gaseous sterilant, and wherein sterilizing the medical device involves passing the gaseous sterilant through the removable cover.

18. The method of claim 16, wherein the gaseous sterilant includes one or more of ethylene oxide, vaporized hydrogen peroxide, ethyl alcohol, nitrous oxide, or nitrogen dioxide.

19. The method of claim 11, wherein positioning the medical device within the exposed cavity comprises aligning a portion of the medical device with a depression in a bottom portion of the tray.

20. The method of claim 11, wherein the retainer includes a dome, and wherein positioning the retainer over the medical device causes the dome to abut against an upper portion of the medical device.
